(19) United States
(12) Patent Application Publication
(10) Pub. No.: US 2021/0085912 A1
(43) Pub. Date: Mar. 25, 2021

(54) Virtual Sleep Environment

(71) Applicant: ROBERT W. SWEETMAN II, CHULA VISTA, CA (US)

(72) Inventor: ROBERT W. SWEETMAN II, CHULA VISTA, CA (US)

(21) Appl. No.: 16/575,446

(22) Filed: Sep. 19, 2019

(51) Int. Cl. A61M 21/00; G06K 9/00; G06F 3/01; G06T 19/00; A61M 21/02; H04N 21/414; H04N 21/422

(52) U.S. Cl. CPC A61M 21/0094; G06K 9/00711; G06F 3/011; G06T 19/006; A61M 2021/0022; H04N 21/414; H04N 21/42201; A61M 2021/0066; A61M 21/02

(57) ABSTRACT

A virtual sleep environment with customized multimedia and climate controls is presented. The invention is mainly comprised of six parts. A first part being a sealed pod with resting bed. A second part being a digital multimedia system that includes a wraparound video screen and surround sound. A third part being a cloud service with proprietary website to manage custom user video and sound content across pods. A fourth part being a biofeedback recording system with sensors and interpretation software. A fifth part being a climate control system that regulates pod temperature according to user preferences. And a sixth part being interconnectivity for local and wide area networks. An object of the invention is to enhance rest effectiveness in busy environments. For example, such resting pods can be stationed in corporate lounges, hospitals, athletic locker rooms, study hall, rest stops and airports.

### CROSS-REFERENCE TO RELATED APPLICATIONS

This application claims the benefit of U.S. Provisional Patent Application No. 62/737,815, filed on Sep. 27, 2018.

### FIELD OF THE INVENTION

The present invention generally relates to the field of sleep. More specifically, it relates to an enclosed resting pod that enhances the resting experience.

### BACKGROUND

The first resting chambers open to the general public were introduced in Japan in the late 1970s and many were run as ‘capsule hotels.’ These chambers were personal pods, complete with TV and an alarm clock. Today, capsule hotels (also known as ‘pod hotels’) are enjoying a new wave of popularity around the world. Capsule hotels have also found homes at international airports. U.K. chain Hotel operates pods at London's Heathrow and Gatwick Airports as well as Amsterdam-Schiphol and Paris-Charles de Gaulle. Japan's First Cabin chain, with room sizes dubbed ‘First Class’ and ‘Business Class,’ opened in Osaka in 2008, expanded to Tokyo-Haneda Airport in 2012, and now boasts eight locations around the country. While such resting chambers have been growing in popularity, few are available in public places for hourly napping that focus on the use of visual and audio technologies for extreme relaxation. Some in the sleep industry have begun incorporating technology to improve rest in such pods. U.S. Pat. No. 8,337,385B1 disclosed a relaxation chamber with coordinating light and sound frequencies but does not incorporate internet connectivity. International Patent No. WO2018033837A1 teaches of a Sleep-inducing chamber with controlled vibrations and sounds but does also not incorporate internet connectivity. United States Patent No. US20160287837A1 disclosed a resting pod for sleep and security that includes biofeedback mechanisms for aiding users in the inducement of sleep but does not include custom user scenarios. United States Patent No. US20190069685A1 disclosed a sleep chamber that includes a power-supply, vents, fans, speakers and lights but does not incorporate sleep sensors. The current disclosure promotes a four-phased process to achieve relaxation that includes critical calm, virtual sunset, sensory deprivation and virtual sunrise.

### SUMMARY OF THE INVENTION

The device herein disclosed and described provides a solution to the shortcomings in the prior art through the disclosure of an enclosed resting pod. An object of the invention is to control and/or modify environmental variables to create ideal conditions for optimal sleep performance. Such performance takes into account natural sleep/wake circadian rhythms. The resting pod accommodates up to two individuals and has a bed, a sealed enclosure (to eliminate interference from external light and sounds) with sound proofing, and a storage area for personal items securely. Such pods can be installed in crowded venues such as airports and train stations without requiring excess space. In addition, the invention can be used at sleep clinics, corporations, hospitals, sports centers, universities and in homes. It can be used for a 20 minute nap or an eight-hour sleep period—or any variation intervals in between (polyphasic sleeping). The sleep pod has the ability to run customized sleep periods with reminders and alerts.

Another object of the invention is to allow a user to attain a deep level of rest within a limited time frame. The invention includes an internal, wraparound, video screen with embodiments having a 1 mm thin, flexible display capable of controlling blue light and turning off every pixel for zero light pollution and a surround sound audio system allowing a user to immerse themselves in a virtual environment that includes a preset timer. The invention also uses ‘biofriendly’ lights to simulate an actual light cycle via a virtual sunset and a scene on the display—such cycles can be relaxing to users' psyche and audio uses specific frequencies to entice the brain to rest.

Another object of this invention is to allow users to customize their resting experience by selecting pre-generated, rest scenarios on a proprietary cloud network service or via a participating third party. The resting pod visual and audio systems are connected to the internet and users can download preselected scenarios from outside vendors and store them on the cloud network. For example, a user preparing for a long trip at home prefers the sounds of waterfalls set to panoramic jungle scenery and imports a scenario from a third party onto the invention's proprietary website on the cloud server. When the user arrives at the airport and is waiting their flight, they can log into the resting pod system, select their customized scenario and begin their customized resting experience.

Another object of this invention is to allow users to customize their resting experience by creating their own rest scenarios and storing them inside a ‘user profile.’ The invention's multimedia experience is designed to follow basic guidelines for good sleep health—that is, specific forms and combinations of light, sound and temperature effect the brain, even during sleep and the multimedia within the invention can optimize these conditions for maximum rest. Within each user profile, each of these settings can be preconfigured. When the user arrives at a sleep pod, whether at home or in public, the sleep pod can quickly adapt to those settings for a customized experience. The user has the ability to adjust the following environmental parameters as part of a profile:

Virtual environment location—Virtual environments are displayed on the wrap around video screen. This imagery creates a video that encompasses the majority of the users' visual-range. Because the invention uses computer generated imagery, the environments look like an actual location or are objects. For these reasons, the user may have a favorite location or a scene that is more calming for their sleep experience.

Light Hue—Specific light hues can influence users' ability to rest. Users have the ability to adjust the light hue within the sleep pod for the optimal experience.

Virtual Sunset speed—During the user's session, the scene will use a virtual sunset to reduce the light within the sleep pod. The rate at which this sunset occurs is based on the length of the sleep period but can be changed by the user to be slower or faster. A slower virtual sunset leaves more light in the sleep pod for longer durations. A faster virtual sunset reduces the light in the sleep pod much faster. The end state of the virtual sunset is complete darkness.

Stars and/or Moon—The user has the ability to turn stars and/or moon on or off during the sleep period. Pitch black is ideal for sleep however many users prefer a dim glow for comfort.

Temperature—The sleep pod is set to reduce the temperature inside the pod by 15 degrees from the mean daytime temperature. If the temperature is 70 degrees external to the pod, this can mean a 55 degree sleep experience. This may be too cold for some users and there is an option to control how cold the experience will get. The threshold for minimum and maximum temperature can be set by the user.

Ambient Sounds—There are ambient sounds within each virtual environment. For example: a beach scene may have waves crashing in, birds chirping, wind blowing etc. Each of these sounds can be reduced or increased for comfort. As the environment transitions to virtual night, the sounds will change. Perhaps birds will be replaced by crickets. These sounds can also be adjusted or turned off.

Binuaral audio—Specific audio frequency patterns can be used to induce brain wave activity. The sounds associated with deep-sleep and dream-sleep can be selected and played in the background. The volume of these passive audio waves can be adjusted for the comfort of the user.

Sleep Period—The user has the ability to set the length of the sleep period. The sleep period is ended by a virtual sunrise that matches the speed and light levels of the virtual sunset, only in reverse. If the user selects a short sleep period and a slow virtual sunset, it is possible that the sunset would never reach full darkness before returning to its sunrise. Warning messages will display when users reach these extreme settings.

Virtual Sunrise—The virtual sunrise is used to wake up the user at the end of the sleep period. Usually, the virtual sunrise is the reciprocal of the virtual sunset, but it too can be modified. Mild light and temperature may not be enough to wake the user, so the user may want the light and temperature to increase much higher than the initial levels.

Another object of the invention is to provide a means to allow users understand the level of rest the invention provides. A biofeedback system inside the pod measures breathing rates, heart rate and body movements and is made available to the user when they log into the website and cloud network via a desktop computer, tablet or smart phone device. The software algorithms analyze the information to determine the level of rest that has taken place (REM sleep etc.). Anonymous forms of the secured data can also be extracted and used for research purposes.

Another object of the aforementioned invention is to provide a means to allow users to adjust the climate within a resting pod. The pod includes a heater and an air conditioning unit that can be adjusted and even preprogrammed to sync with the aforementioned, customized video and audio scenarios. For example, those who prefer a tropical climate can raise the temperature to simulate a sunny warm environment. In addition, it is important to note that there are optimal temperature levels conducive to restful sleep and the invention takes these parameters into consideration. When simulating circadian rhythms, the pod will drop by at least but not limited to 15 degrees from the mean daytime temperature as the light phases out. Upon ending the sleep experience, the light and temperature increase for a virtual sunrise.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows a top view and side view of the invention.

FIG. 2 shows a perspective view of the invention with cover open and closed.

FIG. 3 illustrates a section view of the invention.

FIG. 4 illustrates a user interacting with the invention.

FIG. 5 illustrates a representative view of the invention process.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure.

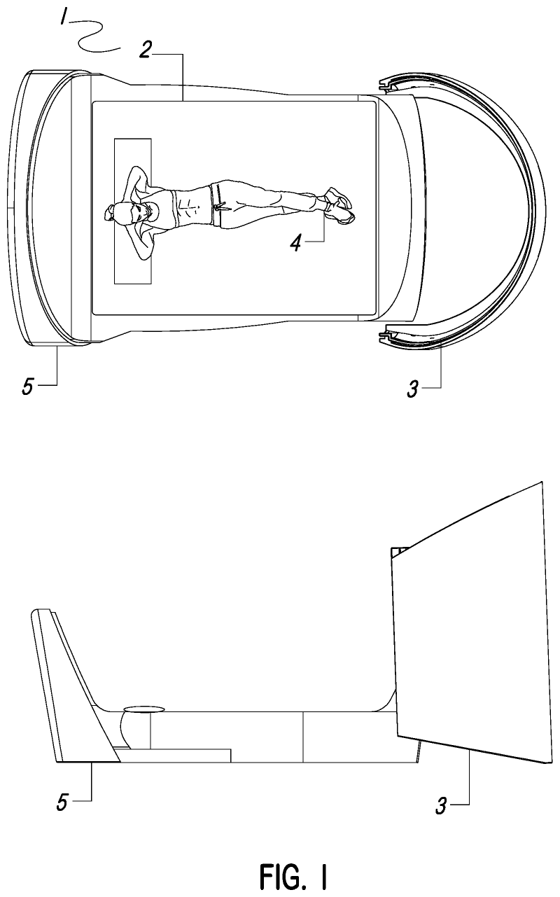

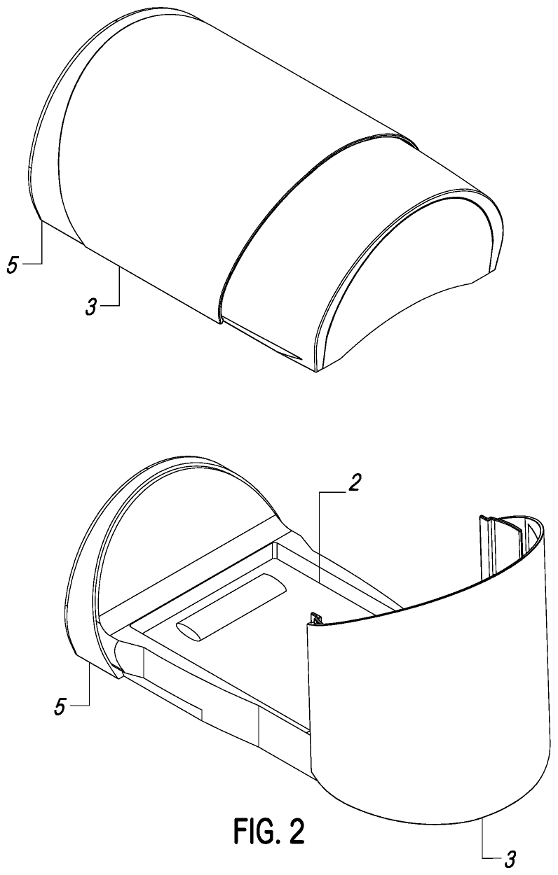

FIGS. 1 and 2 showing a top view, side view and perspective views of the preferred embodiment of the invention 1 with slidable cover 3 open and closed. The figures showing the invention being a rectangular-shaped pod with said cover 3 and enclosure 5 being made of a rigid material such as but not limited to metal, plastic, fiberglass and the like and containing a resting portion 2 (comprised of a bed etc.) configured along its longitudinal axis. The invention also being configured to receive a resting user 4 therein. Said cover 3 being controlled manually by a user with handles thereon. A secured storage area is included on a distal portion to allow for personal belongings storage.

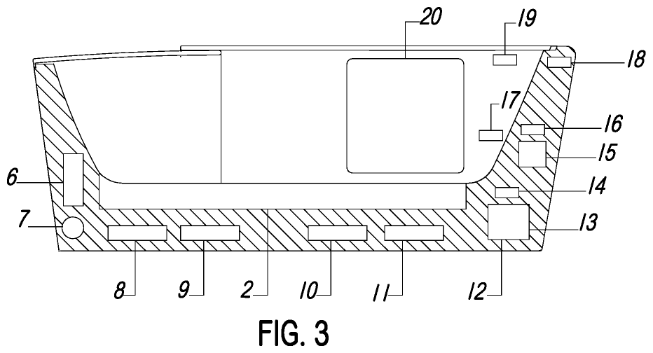

FIG. 3 illustrates a section view of the invention showing internal systems that include, but may not be limited to: an onboard widescreen video display 20 (such as OLED, LCD etc. having ratios of at least 3:1); speakers with stereo, surround sound (Dolby etc.); onboard specialty lighting and effects 19 (LED etc.); an onboard logic controller 16; an onboard central processing unit (CPU) or micro controller 13 with onboard memory, multimedia controllers, WiFi 14 and onboard cellular 16 transmitters and the like; onboard biofeedback sensors (open 9, closed 11, ballistocardiograph 10 and millimeter wave sensor 17 etc.); onboard climate controls 6 regulating hot and cold air (such as a rechargeable coolant system and an electric, radiant heating elements) and having a fan and air return plenum. The aforementioned OLED technology having the ability to turn each individual pixel off which allows the invention to control blue light and eliminate light pollution. All the aforementioned systems being connected to and controlled by said central processing unit as well as to an onboard A.C. power supply 8 therein. The aforementioned CPU 13 having software being written from code that may include, but not be limited to: Java, C++™, Visual Basic™, Linux™, Basic™ and the like. The software also compatible with a plurality of operating systems such as, but not limited to: Windows™, Apple™, and Android™, and compatible with a multitude of hardware platforms such as, but not limited to: personal desktops, laptops, tablets, smartphones and the like.

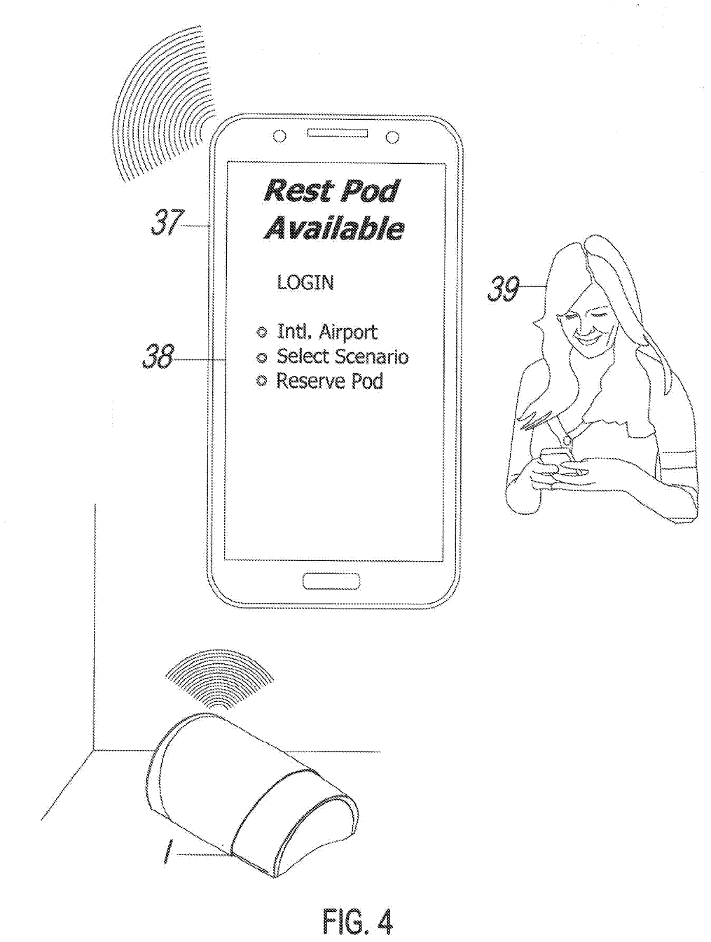

FIG. 4 illustrates a user 39 interacting wirelessly with a resting pod 1 by means of mobile device software application 38. Said software also containing a marketplace for developers around the world to submit ‘virtual environments,’ whereby they offer unlimited relaxation experiences for a user. The figure showing the user interface allowing a for functions such as but not limited to selecting a resting pod location, a preset relaxation scenario to be downloaded to resting pod 1 and reserving a time to utilize said resting pod 1. The mobile device software application 38 being written from code that may include, but not be limited to: Java, C++™, Visual Basic™, Linux™, Basic™ and the like. The software also compatible with a plurality of operating systems such as, but not limited to: Windows™, Apple™, and Android™, and compatible with a multitude of hardware platforms such as, but not limited to: personal desktops, laptops, tablets, smartphones and the like.

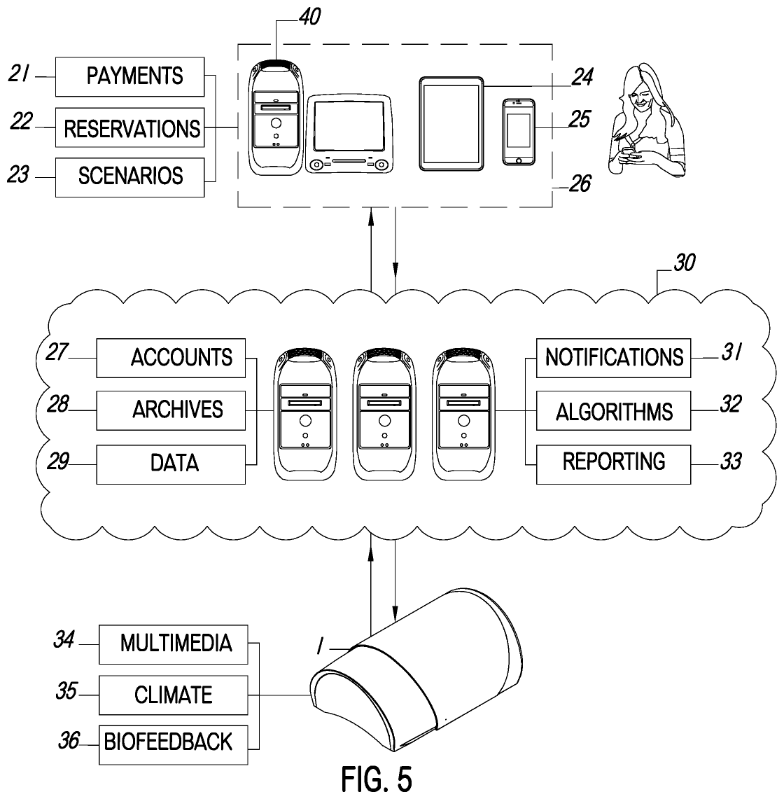

FIG. 5 illustrates a representative view of the invention process wherein the resting pod 1 having processes such as but not limited to: receiving and sending multimedia files 34 representing preset resting scenarios; climate control 35 settings that can be synced to resting scenarios; and biofeedback 36 files. Said multimedia files may be selected from a user's profile and may include but not be limited to adjusting and setting the following parameters: virtual environment location, light hue, virtual sunset speed, stars and/or moon, temperature, ambient sounds, binuaral audio, sleep period and virtual sunrises.

The figure also showing users having the ability to perform functions such as but not limited to making online payments 21 for using resting pods (paypal, venmo etc.); making reservations to use a resting pod 22 and having capabilities to integrate with existing online calendars (google calendars etc.); and select from a range of pre-generated rest scenarios 23 (beach scenes, woodland areas etc.). Users having the option of using computing devices such as but not limited to tablets 24, cell phones 25 and desktop computers 40.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

What is claimed is:

1. A resting pod comprised of the following parts:
(a) a selectively-sealed chamber;
(b) onboard climate controls;
(c) bed portion;
(d) onboard multimedia;
(e) onboard CPU;
(f) onboard biofeedback sensors;
(e) software; and
(f) a cloud network.

2. The resting pod of claim 1 wherein the selectively-sealed chamber having a slidable cover.

3. The resting pod of claim 1 wherein the onboard climate controls include a cooling system and an electrical heating system.

4. The resting pod of claim 1 wherein the onboard multimedia includes a wraparound video screen and surround sound speakers.

5. The resting pod of claim 1 wherein the onboard CPU containing WiFi and onboard cellular transmitters.

6. The resting pod of claim 1 wherein the onboard biofeedback sensors include a ballistocardiograph and a millimeter wave sensor.

7. The resting pod of claim 1 wherein the software including a mobile device application.

8. A method for enhancing rest, the method comprised of the following:
(a) reserving the rest pod;
(b) selecting the rest scenario;
(c) resting inside the closed pod;
(d) being exposed to preferred visual and auditory stimulation;
(e) recording biofeedback; and
(f) analyzing biofeedback.

9. The method for enhancing rest of claim 8 wherein the selecting the rest scenario being performed on the cloud network of claim 1.

10. The method for reserving a rest pod of claim 8 wherein the reserving being conducted on integrations with users' existing online calendars.

11. The method for enhancing rest of claim 8 wherein the visual and auditory stimulation being selected in the rest scenario and settings in the user profiles.

12. The method for enhancing rest of claim 8 wherein the recording of biofeedback including the recording of heart rate, body movement and breathing rates.

13. The method for enhancing rest of claim 8 wherein the analyzing of biofeedback determining quality and type of sleep.

14. The method for enhancing rest of claim 8 wherein being exposed to preferred visual and auditory stimulation allows light levels to communicate with the retina and consequently the suprachiasmatic nucleus.

15. The method for enhancing rest of claim 8 wherein being exposed to preferred visual and auditory stimulation allows sound frequencies that reduce or increase brainwave activity

16. The method for enhancing rest of claim 8 wherein the resting inside a pod includes exposure to temperature levels that signal the hypothalamus that nighttime has arrived, and the body can begin the sleep process.
